(19) United States
(12) Patent Application Publication
(10) Pub. No.: US 2026/0363585 A1
(43) Pub. Date: Nov. 19, 2026

(54) DISPOSABLE RF SENSOR FOR DIELECTRIC CHARACTERIZATION OF BIOLOGICAL MATERIAL

(71) Applicant: SUR'TOUCH LTD, Oxford Oxfordshire (GB)

(72) Inventors: Thierry DESJARDINS, VALBONNE (FR); Nicolas VEAU, LE ROURET (FR)

(73) Assignee: SUR'TOUCH LTD, Oxford Oxfordshire (GB)

(21) Appl. No.: 19/477,434

(22) PCT Filed: Apr. 21, 2023
(86) PCT No.: PCT/EP2023/060474

§ 371 Date: Oct. 21, 2025

(51) Int. Cl. A61B 5/1477; A61B 5/1495; A61B 10/00; A61M 1/00

(52) U.S. Cl. CPC A61B 5/1477; A61B 5/1495; A61B 10/0045; A61M 1/87; A61B 2505/05

(57) ABSTRACT

A tissue or liquid characterization sensor modules improves the surgery process by eliminating downtime associated with characterizing abnormal tissue or liquid in an area under surgery. The sensor module automates the characterization process so that surgery is not delayed by the need to characterize a localized issue or tissue that is present in the area. The sensor module is connected to a database of characterization files which are referenced when a signal on tissue or liquid being characterized is returned and matched to one of the files. Thus, characterization becomes instantaneous eliminating the need for manual characterization before the surgery resumes.

### BACKGROUND

The embodiments herein relate generally to medical equipment.

Since the beginning of surgery history, very few innovations in the handheld equipment have been provided to help surgeons in their tasks. Yet, the surgeon must apply increasingly complex procedures to treat patients, in less time, with always higher success rate to respect health insurance requirements.

In some scenarios, surgery includes the presence of abnormal tissue. FIG. 8a shows a conventional procedure employed by surgeons. The abnormal tissue that must be removed is sufficiently visible with modern imaging equipment, but as the surgeon progresses along through the surgery, the image becomes incorrect and at some point, irrelevant because the tissue self collapses rendering the image worthless. In some instances, the patient must be closed up if a localized issue occurs before resuming the surgery. If the tissue needs to be characterized before continuing surgery, the surgical procedure may be delayed. The surgeon has only his instinct and in-depth knowledge of anatomy to continue the surgery and remove what is supposed to be the abnormal tissue.

Embodiments of the present invention address these problems.

### SUMMARY

In one aspect of the subject disclosure, a tissue or liquid characterization sensor module for surgery comprises a rear end of the module connected to a vacuum source; a front end of the module configured to gather tissue or liquid to be characterized, wherein the tissue or liquid to be characterized is drawn into the module by the vacuum source; an RF signal source configured to generate an RF signal transmitted to the tissue or liquid to be characterized; an antenna configured to receive a reflection signal from the tissue or liquid to be characterized; and a characterization module configured to match the reflection signal to a known file in a database.

### BRIEF DESCRIPTION OF THE FIGURES

The detailed description of some embodiments of the invention is made below with reference to the accompanying figures, wherein like numerals represent corresponding parts of the figures.

FIG. 1A is a schematic view for a process of dielectric characterization using a radio frequency (RF) sensor according to embodiments of the subject disclosure.

FIG. 1B is a block diagram of an RF sensor according to an embodiment.

FIG. 2 is a perspective, top internal view of a sensor according to an embodiment.

FIG. 3 is a hybrid view of interdigitated electrodes in a sensor according to an embodiment.

FIG. 4A is a diagrammatic view of a circular array of sensor structures according to an embodiment.

FIG. 4B is a diagrammatic view of a matrix array of sensor structures according to an embodiment.

FIG. 5 is a diagrammatic view of an impedance measurement for a sensor according to an embodiment.

FIG. 6A is a flowchart for a method of manufacturing a sensor according to an embodiment.

FIG. 6B is a flowchart for a method of operating a sensor according to an embodiment.

FIG. 7A is a flowchart diagramming material and information flow during operation of a sensor according to an embodiment.

FIG. 7B is a diagrammatic view of a signal processing flow according to an embodiment.

FIG. 8A is a flowchart of a method for conventional surgery.

FIG. 8B is a flowchart of a method of surgery that provides instantaneous local characterization of an area being treated according to an embodiment of the subject disclosure.

FIGS. 9 and 10 are a block diagram of an architecture of a system for dielectric characterization of a surgical site sample using a radio frequency (RF) sensor according to an embodiment.

FIG. 11 illustrates the subcutaneous detection according to an embodiment of the invention.

FIG. 12 is a graph showing results for the embodiment of FIG. 11.

FIG. 13 is a block diagram of an embodiment according to the invention, pertaining to electroporation support.

FIG. 14 illustrates the simultaneous detection and mapping according to an embodiment of the invention.

### DETAILED DESCRIPTION

Embodiments of the subject technology provide a low footprint sensor which has the ability to discriminate between normal tissue and abnormal tissue. In one aspect, the sensor disclosed includes a per-operative real-time analysis of human constituents. The sensor is seamlessly integrated into surgery instrument, keeping the learning curve low and augmenting surgery instrument capabilities. As a result, actionable and practical information is instantaneously available to surgeon. As will be appreciated, the sensor is disposable (unlike classic sterilizable devices). The sensor is merged within regular smart ancillaries vs. classic inert ancillaries. In a system embodiment, data may be collected from the RF response of a tissue following an RF stimulation which is an improvement over classic optical characterization that merely relies on looking through a microscope. Some embodiments include automated dimensionality reduction based on database of results which improves on the classic manual process used by an anatomopathologist. The database is re-enforced by gathering data from previous surgeries.

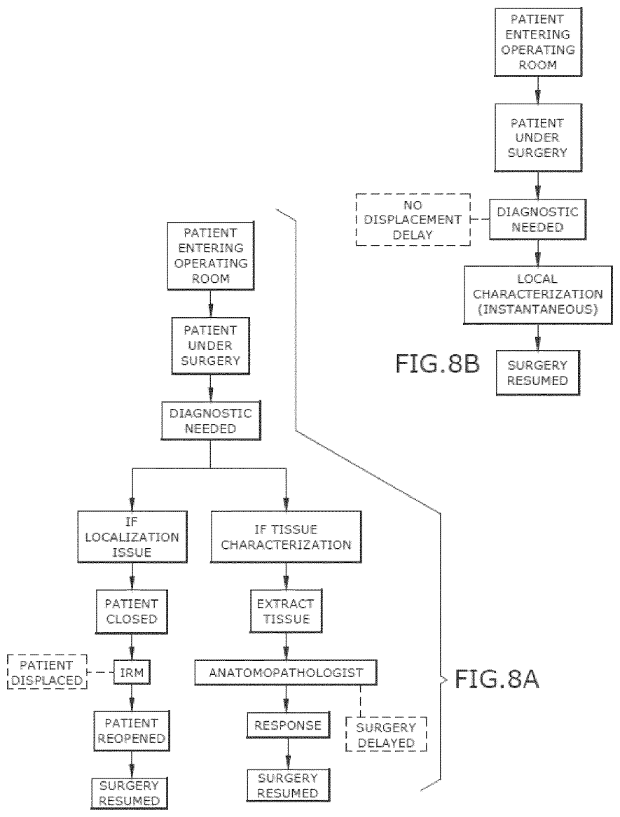

FIG. 8B illustrates a surgical process flow using a sensor according to aspects of the subject disclosure. As may be appreciated, in comparison to the conventional flow shown in FIG. 8A, aspects of the subject disclosure streamline the surgical process and may eliminate the need to pause surgery for tissue characterization or localized issues, because a sensor embodiment provides instantaneous local characterizations of the area under surgery. Aspects of the subject technology may analyze a sample at the surgical site and provide an indication for example of whether the sample is normal or abnormal in real-time and on the spot (as compared to the conventional process of sending the tissue sample out for testing).

Referring now to FIG. 2, a sensor device 10 is shown according to an exemplary embodiment. The sensor device 10 includes for example, an electronic module 12, an antenna module 14, an energy source 16, and a transfer module 18. In the case of liquid characterization, the back end of the device 10 may be connected to a vacuum source (for example, an operating room vacuum source or syringe), and the front end is in contact with either the liquid or the tissue sample to characterize. The electronic module 12 includes a circuit of elements which provide the transmission and receipt of a signal, comparison of the return signal to stored data, and comparison logic to determine whether the return signal indicate a normal or abnormal tissue sample. The details of the circuit are described farther below. The antenna module 14 may be part of the electronic module 12 but is generally a section of electronics which includes the receiving component of the reflected signal used for characterizing the tissue sample. The energy source 16 may be a battery, capacitor, or connection to a wall socket which provides the power for the electronic module 12. The transfer module 18 may be housing through which the tissue sample passes through. The transfer module 18 may be the point of analysis for the electronic module 12 and antenna module 14 to analyze the tissue sample as the tissue sample passes from the front end to the back end and out of the device 10.

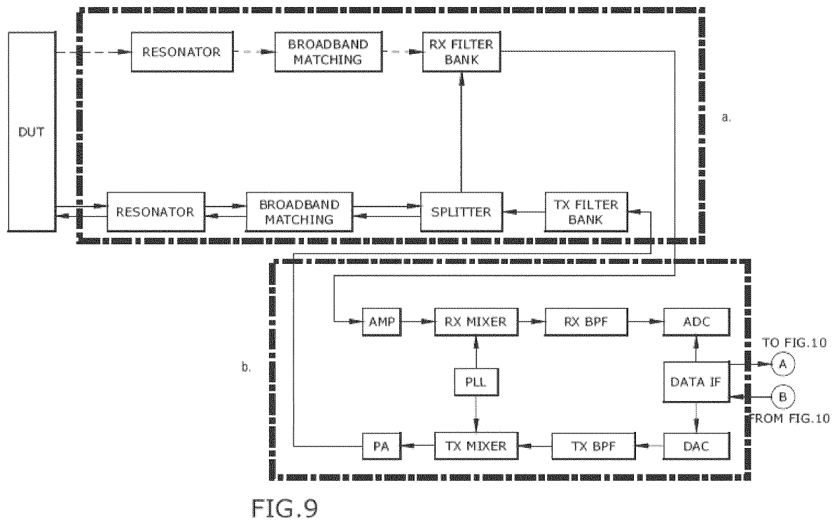

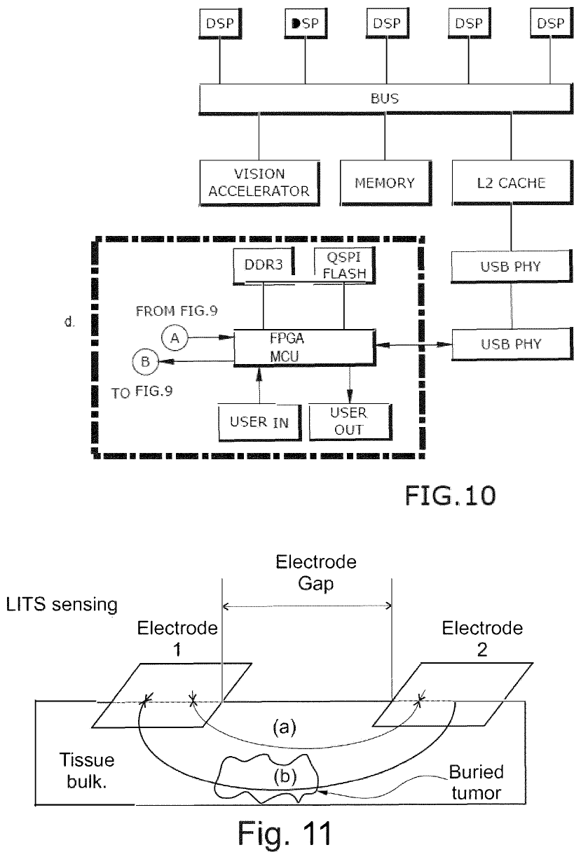

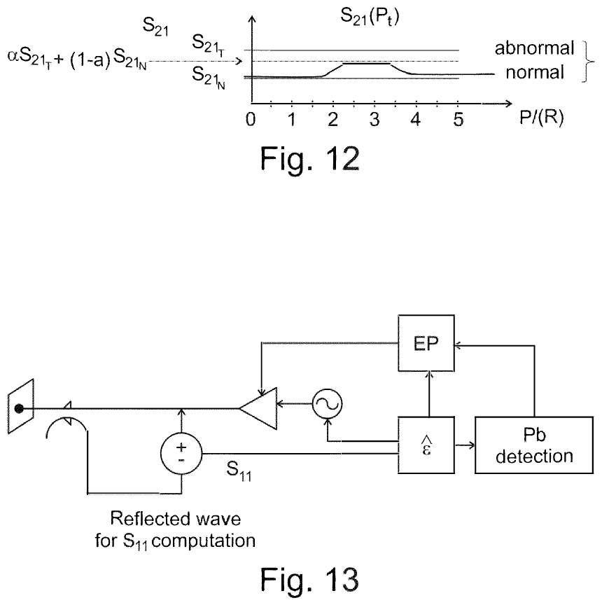

FIGS. 9 and 10 show an architecture of the device 10 in more detail according to an exemplary embodiment. The architecture is broken up into blocks, (a.), (b.), (c.), and (d.), with blocks (a.), (b.), and (d.). being outlined and the elements of block (c.) without outline in FIG. 10. The device 10 may include a comparison module which may comprise an application specific integrated circuit such as a DSP, a TPU (c). Operational programming may be run on a general purpose microcontroller (MCU), assisted by a specific encoder implemented on the FPGA that formats RF data for the comparison module and transfer them through a high bandwidth interface, buffered with a fast access dedicated memory. A database of stored normal and abnormal signal frames may be stored in flash and transferred at runtime in the comparison module through a cache hierarchy made of fast access memory. The device 10 also include a frequency conversion module (b), where a specific low frequency signal is synthetized by the FPGA and mixed with the local programmable oscillator to produce the high frequency wave.

This wave is designed to interact with (a) the resonator electromagnetically coupled with the biological material over a large frequency range and to produce a significantly different reflected waves for the two or more categories under considerations.

The reflected signal is synchronously mixed with the same programmable local oscillator, transposed in a lower frequency and analyzed by the comparison module.

An antenna module section may include a phase locked-loop control system for generating the signal that is transmitted to the tissue sample (shown as the device under test “DUT”). The signal reflected by the tissue sample may be transmitted to the FPGA which may compare the reflected signal frame to stored signal frames in the memory.

As will be understood, the sample may generally be at the site of s surgical procedure (for example, on a patient's body), however some samples may be displaced from the patient but may be generally nearby the surgical procedure. The user (for example, a surgeon or robotic arm) may hold the device 10 between his two fingers like a pen. One of the fingers starts the sensor module by a pneumatic or electric switch. As will be appreciated, an answer from the device 10 is generated almost instantaneously (for example, within 1 second). The indication of a normal or abnormal sample may be provided by a sound and/or a visual indicator coming from the device 10.

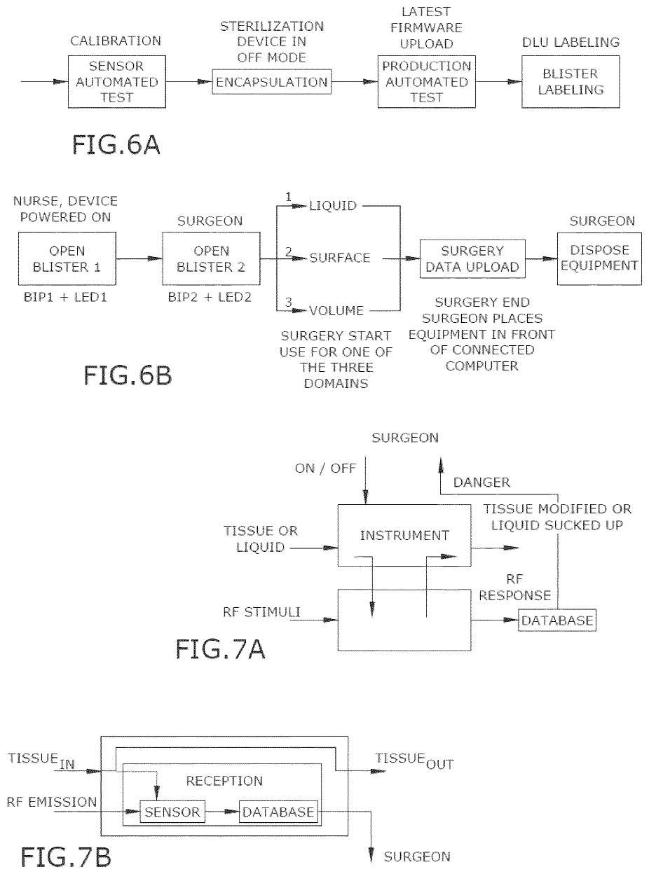

FIG. 6A shows example manufacturing/engineering processes to produce the sensor module. FIG. 7B also shows operation of the sensor module in an exemplary embodiment. The biological material in front of the device is transferred to the sensor antenna inside the device by a pneumatic or hydraulic microfluidic module or by a simple mechanical action. The electrical signal provided by the sensor module is altered by the material in front of the antenna. The structure is composed of an embedded computing unit (for example, a processor or logic circuit) and its microprogram. This unit processes a succession of identical frames (sequence of samples) coming from the transmitted RF signal reflected by the tissue, then collected by the sensor antenna and converted into frames. In the computing unit, each frame is compared with a set of reference frames stored in an embedded memory, previously categorized in few classes by a committee of medical experts and automated classifiers, typically as “healthy”, “unhealthy”, “out of range” classes. The probability of a frame to belong to a particular class is given by the distance between the measured frame and the reference one. This probability is finally converted into a hard decision directly visually or audibly interpretable by the practitioner. No learning curve is necessary because the device automates the tissue characterizations by analyzing the tissue content and referencing the database.

FIG. 6B shows an example of material and information flow. The device is ready to use when removed from the sterilized blister (automatically powered on and auto checked) when it is in contact with air. Human material is brought in front of the sensor as described above. The sensor injects a multi-frequency RF stimulation in the human material. Simultaneously the sensor receives an RF reflection/transmission corresponding to the RF response of the human material. FIG. 5, shows an example of impedance measurement for the RF response of a tissue sample. This RF response is compared to a pre-recorded database. See for example, FIGS. 7A and 7B which show a database referenced after a reflected RF signal generated by the tissue sample is captured. Depending on the matching confidence level, the information returned to the surgeon is material abnormal, material normal or out of range. After surgery, the device wirelessly sends anonymized RF response collected during the surgery to a centralized database for decision reinforcement, and can be disposed. The following described specific applications using embodiments of the sensor module, modified if necessary where applicable.

Liquid analysis: The modified cannula with RF sensor transfers the liquid from the inlet to the sensor via a debit controlled fluidic mechanism that maintains fluid speed and particle size within the limits (the human material and biological liquid present in the operating field are drawn up from the cannula inlet to the vacuum source to which the back of the equipment is connected.). Thanks to the vacuum source, the liquid transfer is actuated by the vacuum source available in every surgery room. While the liquid is transiting, it will be in contact with the surface of the RF antenna and radiated with RF microwaves for which their reflections will be altered by the characteristic impedance of this material.

Surface analysis: If we want to detect the limit of a surface alteration of the tissue, as is the case in dermatology, the sensor has to scan the surface of the organ to diagnose. The operating mode is to bring the head of the handheld equipment to the point to be probed. On the head of this handheld device resides a small RF antenna working in reflection mode. A few other ones could surround this antenna in order to increase the spatial selectivity of the sensor. In addition, the spatial localization could be improved by addition of an optical sensor sensitive to the lateral displacement.

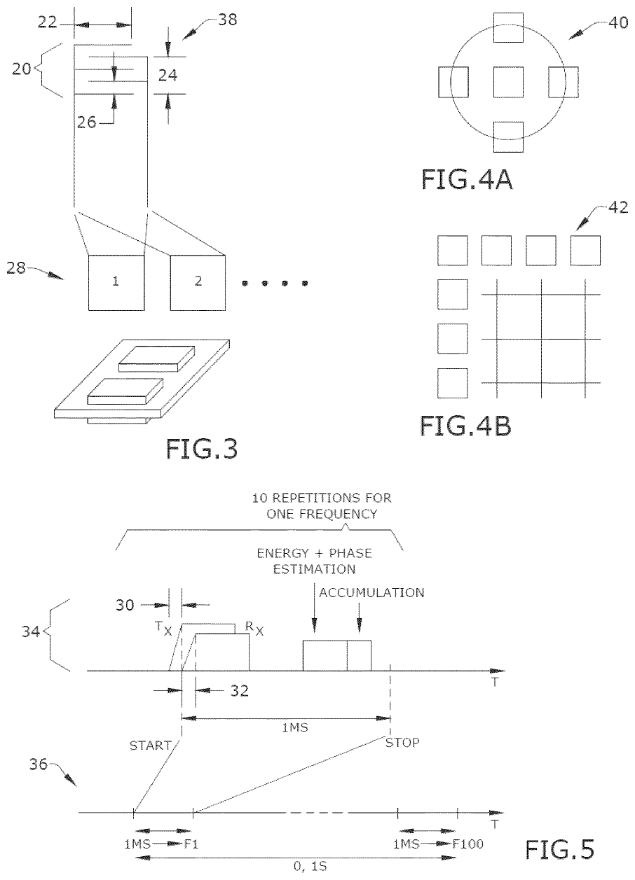

Volume analysis: If we want to detect the boundary of the alteration of the tissue, as is the case for breast cancer for example, the sensor has to be inserted into the organ and be able to sense a difference in the tissue characteristic between both sides of the sensor. See FIG. 3 for an example of an embodiment which includes two interdigitated electrodes (IDEs) 28 for volume measurements. So, the sensor is mounted on each face of the cutting blade. Both sides of the sensor will work in reflection mode. The antenna generates an electrical field in close proximity of the sensor by, applying a variable difference of potential between the two disconnected set of fingers. The energy stored by the electrical field is controlled by the distance between the two sets of fingers (26: the finger gap) and the unfolded length of the fingers given by the height (24), length (22) and numbers (20). In a first approximation, the dielectric properties of the material facing the electrode modifies the amount of energy stored at each frequency, and consequently the reflected energy. FIGS. 4A and 4B show IDE configurations according to other embodiments of the sensor module. The IDE arrangements provide improvements in characterization measurements depending on the application they are used for. Linear arrangement allows consecutive acquisitions to perform an S21 measurement or an optical flow measurement, best suited for liquids. The double side arrangement allows differential S11 measurements during the tumor ablation, best suited to discriminate the boundaries between healthy and malignant area. The planar matrix configuration allows 3D measurement steering, best suited for buried malignant area. The circular matrix allows a 2D special measurement steering, best suited for skin surface anomaly detection.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

A variant of the cannula sensor can be built to monitor the healing process after the surgery in the form of a capsule and a reader permanently embedded inside the patient body.

Clinical reasons: After the tumor removal and depending on the extend of the surgery, it exists a risk of recidivism worth tracking.

Design evolution: The two mechanisms of sensing and wireless energy transfer rely on the same electromagnetic resonance principles, allowing a sensor configuration with no physical connection with the signal processing and therefore, well-suited for a minimalistic passive standalone implantable sensor.

Use case: We want to perform dielectric measurements after the surgery without new surgical intervention.

In order to do that, we have to place a miniature implantable passive transponder inside the patient body, with dielectric sensitive back-scattering capabilities.

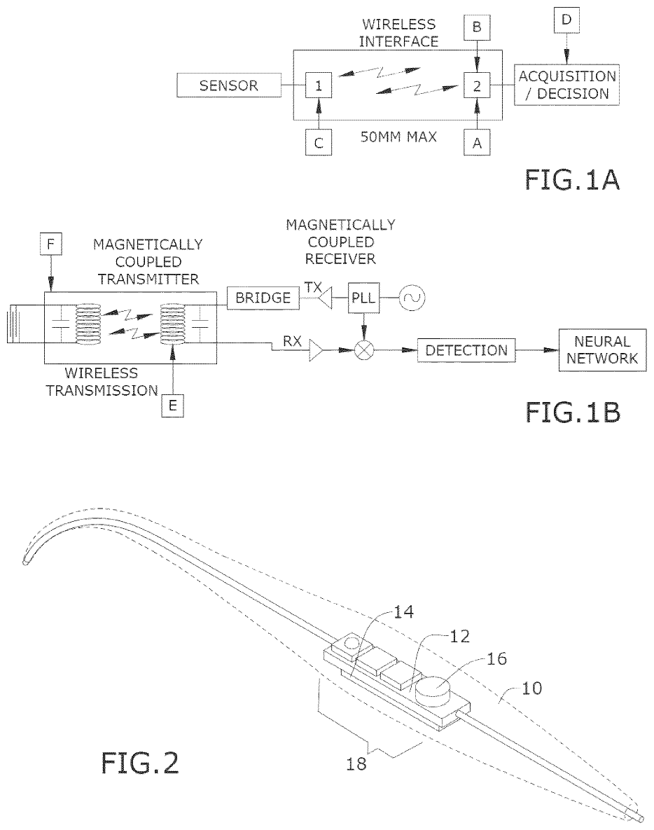

The principle relies on:
A forward power: FIG. 1A, “A”. Interface (box “1”) inductively transfers power from handheld equipment. If power is applied inadvertently, no hazardous induced current can be generated.
Forward telemetry: FIG. 1A, “B”. The sensor is activated when a frequency specific forward power is applied within 10 cm of the sensor The sensor stops working when no power is applied
Backward telemetry: FIG. 1A, “C”. The sensor interface (box “1”) back scatters according to the nature of the dielectric around the sensor. The interface analyzes the received information.
Processing, which is the same as for cannula, FIG. 1A, “D”.

FIG. 1B, “E”., the implementation of the capsule in the patient body uses the same dielectric resonance principle as for the sensor in the cannula, but scaled down, and it is passive, which means there is no battery, no hardware and no software.

On the structure side, FIG. 1B, “F”., the implanted capsule is smaller than the cannula sensor, it is made of bio-compatible monolithic component and there is no moving mechanical part, so no encapsulation is needed.

Afterwards, the implanted sensor can be transcutaneous activated when necessary.

The benefits of such a variant are to monitor as closely as possible the evolution of the tissues in order to leverage the initial surgery.

### Subcutaneous Detection

The system can detect tumors buried below the surface up to 2 mm deep, by using the variation of the transmit power and the comparison of measured values.

As illustrated in FIG. 11:
(a) For a weak emission signal of the S21 parameter, the electric field lines created between the two electrodes remain above the tumor and are not affected by the presence of the tumor.
(b) For a strong emission signal measuring the parameter S21, the field lines extend deeper than in the previous case and the parameter S21 is subject to changes related to the presence of the tumor.

This dependence of the depth of detection on the transmitted power is amplified thanks to a particular shape of the electrodes that promotes a physical effect called edge effect.

The higher the measurement signal for the S21 parameter, the more the system will be able to detect deeply buried tumors.

The sensitivity of the instrument to depth sensing depends on the 3D shape of the antenna, summarized by the alpha discrimination factor.

To obtain a significant variation, indicating the presence of a tumor leaked at a depth of 2 mm, a variation of the parameter of 10 dB compared to the absence of tumor is required.

If we consider the relative permittivity of tissues between 5 and 60 epsilon0 and an interdigitated electrode of 100 elements with a 5 um gap and a surface of 1 mm2 per electrode, then alpha should be higher than 80. The proposed configuration of the electrodes figures out an alpha of 0.85.

### Electroporation support

In addition to diagnosing tumors, the invention can help treat them by electroporation.

Indeed, the collected dielectric information may be used to calibrate the electric field responsible for electroporating the cells.

The modes of operations are described below, as illustrating in FIG. 13:
1. Automatic initiation of electroporation during tumor detection upon surgeon authorization
2. Automatic adjustment of the frequency, time sequence, power of the electric electroporation field according to the dielectric nature of the underlying tissue.
3. The electroporation exposure time of electroporated tissue is kept under a maximum safe value by aggregating the individual exposures on the self-generated map.

### Simultaneous Detection and Mapping

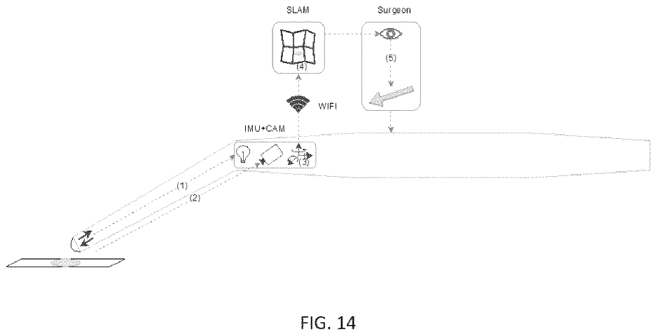

The device may detect and warn the surgeon of the viability of the tissue, as illustrated in FIG. 14.

Then the surgeon may decide whether to correct the problem right away or continue the analysis. In the latter case the surgeon must remember the position of the problem detected previously.

In this embodiment, to avoid committing to memory the location of the tumor detected but not treated, the system maps all detected positions on an automatically generated 3D map.

The map and the annotations are refreshed each time the cannula rescans the same area.

Then we can guarantee an all-time true representation of the situation, especially useful at the end of surgery when a final scan will confirm that no tumor is remaining.

All the information needed to reconstruct the surgical scene and all the tissue dielectric data needed to assess tissue quality are timely available at the same location.

In the embodiment of FIG. 14, this information is:
(1) The tumor detection timestamp
(2) The images captured from different angles of the surgical scene.
(3) The absolute position and orientation of the cannula.
(4) It makes it possible to build instantly and efficiently an annotated dynamic map of the relevant data.

The cannula software advantageously estimates the horizontal position and altitude of the detected singularity and superimposes this position on a self-generated 3D map by accumulating previous images and accelerations on the different axes.
(5) This information is then transmitted to a fixed device such as a screen, or a wearable such as smart glasses, to allow surgeons to improve their understanding of the situation.

1. A tissue or liquid characterization sensor device for surgery, comprising:
a housing;
a connection in the housing connectable to a vacuum source;
an inlet configured to receive animal tissue or liquid to be characterized, wherein the tissue or liquid to be characterized is drawn into the housing by the vacuum source; a
radio frequency (RF) signal source in the housing configured to generate an RF signal transmitted to the tissue or liquid to be characterized; an antenna configured to
receive a reflection signal from the tissue or liquid to be characterized; and a characterization module configured to compare the reflection signal
to a stored file.

2. The device of claim 1, wherein the characterization module includes a processor configured to compare a transmitted frame of the RF signal to a reflected frame of the RF signal, wherein the reflected frame is generated by the transmitted frame impacting the tissue or liquid to be characterized.

3. The device of claim 1, wherein the characterization module includes two interdigitated electrodes (IDEs), wherein a measurement of the IDEs is used by the characterization to measure a volume of the liquid to be characterized.

4. The device of clam 1, wherein the characterization module includes an array of interdigitated electrodes (IDEs), wherein a measurement of the IDEs is used by the characterization to measure a volume of the liquid to be characterized.

5. The device of clam 1, wherein the characterization module includes planar matrix of interdigitated electrodes (IDEs), wherein a measurement of the IDEs is used by the characterization to measure a volume of the liquid to be characterized.

6. The device of clam 1, wherein the stored file is stored in memory in the device housing.

7. The device of clam 1, wherein the device automatically powers on when in contact with air.

8. The device of claim 1, being configured to detect tumors buried below the surface up to 2 millimeters deep by using the variation of the transmit power and the comparison of measured values.

9. The device of claim 1, wherein the collected dielectric information is used to calibrate the electric field responsible for electroporating the cells.

10. The device of claim 1, being configured to detect and warn the surgeon of the viability of the tissue, the device being especially configured to map all detected positions on an automatically generated 3D map in order to avoid committing to memory the location of the tumor detected but not treated.
